(19) United States
(12) Patent Application Publication
(10) Pub. No.: US 2021/0353799 A1
(43) Pub. Date: Nov. 18, 2021

(54) ULTRAVIOLET (UV) DISINFECTOR

(71) Applicant: Patrick Spears, Brentwood, CA (US)

(72) Inventor: Patrick Spears, Brentwood, CA (US)

(73) Assignee: CleanNesta LLC, Brentwood, CA (US)

(21) Appl. No.: 17/391,030

(22) Filed: Aug. 2, 2021

(51) Int. Cl. A61L 2/10; A61L 2/24; A61L 2/28

(52) U.S. Cl. CPC A61L 2/10; A61L 2/28; A61L 2/24

(57) ABSTRACT

An ultraviolet (UV) disinfector is disclosed. The UV disinfector is configured to automatically deliver an effective dose of ultraviolet radiation for antibacterial and antiviral treatment of various surfaces. The UV disinfector could be operated in different operating modes based on the surface disinfection. The UV disinfector comprises a controller having a processor and a memory in communication with the processor, wherein the memory is configured to store a set of instructions, which are executed by the processor. The UV disinfector further comprises an UV light-emitting diode (LED) array, which is securely affixed to a base section of the housing. The UV LED array in communication with the controller is configured to provide a disinfecting illumination with an ultraviolet light to the exterior surface. A plurality of sensors in communication of the controller is configured to send signals to the controller, thereby controlling the operation of the UV disinfector.

### BACKGROUND OF THE INNOVATION

### A. Technical Field

The invention disclosed herein generally relates to disinfectors. More particularly, the present invention relates an ultraviolet (UV) disinfector integrated with a control system, configured to automatically deliver an effective dose of ultraviolet radiation for antibacterial and antiviral treatment of various surfaces and disinfect enclosed spaces such as hospital rooms, locker rooms, food processing facilities, and other locations where surface disinfection is desired.

### B. Description of Related Art

Ultraviolet (UV) disinfection devices could be used to disinfect enclosed spaces where surface disinfection is desired. UV disinfection devices reduce the use of chemical preservatives and disinfectants. These UV disinfection devices offer the advantage of lower operating costs as compared with conventional chemical additives and cleaning agents. These devices are utilized in many industries as an environmentally safe and regulation free method of sterilization. Additionally, UV disinfection devices are free from consumer or environmental concerns that are often voiced regarding conventional chemical disinfection methods. These devices have been used successfully in many industries, drinking and process water applications, and in hospitals, pharmaceutical and beverage production, etc.

Bacteria, molds, and viruses substantially absorb UV wavelengths of 210 nm and 310 nm. It is understood that this absorbed UV radiation adversely affects the survivability of many pathogens, such as bacteria, molds, and viruses. Standard, commercially available UV lamps could efficiently emit a broad spectrum that includes these specific wavelengths of UV light, and in the intensity required for effective control. However, these UV lamps may be harmful to humans and other life forms and are typically shielded or in environments where exposure is limited.

Few existing patent references attempted to address the aforementioned problems are cited in the background as prior art over the presently disclosed subject matter and are explained as follows:

A prior art US20200206375 assigned to Ufkes; Philip J., discloses a portable UV-C disinfection apparatus, method, and system for ultraviolet germicidal irradiation. A controller is communicably engaged with a UV-C sensors to determine the amount of UV-C radiation collected by the UV-C sensors, and it includes instructions stored on a memory according to the amount of UV-C radiation collected corresponding to an effective kill-dose for surface disinfection. The device is provided with a software with firmware, resident software, micro-code, etc. The data provides information as to the orientation of objects in a room and the time and dosage strength needed to disinfect a room, and it is stored in the portable UV-C disinfection system memory. The UV-C disinfection apparatus is comprised of a left and a right array surface, a left and a right UV-C sensor, a front and a rear proximity sensor, a base housing, a left and a right emitter array and tracks. The user can select an operational mode or configuring a target dosing variable corresponding to a specific group or type of microorganism. The device is engaged with the controller through a wireless communication interface, such as Bluetooth or WiFi.

Another prior art US20210010701 assigned to Nesler; Clay G., discloses a systems and methods for reducing health risks with respect to an infectious disease in buildings. A disinfection subsystem controller is used to control various disinfectant mechanisms; it performs the disinfection action by using disinfectant light to sanitize air circulated through the space in the building or performing air filtration at an air handling unit of the BMS. The UVC emits number of radiation cycles, duration of radiation cycle, and/or current high-threat pathogens, and the healthcare data is used to adjust the disinfection parameters of the UVC (e.g., wavelength, dosage duration, time between cycles, etc.) emitted from UVC source. The Zone controllers can communicate with individual BMS devices (e.g., sensors, actuators, etc.) via sensor/actuator (SA) busses, and it is connected to various sensors (e.g., temperature sensors, humidity sensors, pressure sensors, light sensors, occupancy sensors, etc.), actuators (e.g., damper actuators, valve actuators, etc.) and/or other types of controllable equipment (e.g., chillers, heaters, fans, pumps, etc.). The system communicates with client devices such as user devices, desktop computers, laptop computers, mobile devices, etc. Though the discussed prior art references are useful to some extent for some purposes, these prior efforts sometimes yield a poor efficiency with poor experience to users. Further, the prior arts could not effectively kill bacteria and viruses on the surfaces and are also harmful to humans and other life forms.

Therefore, there is a need for an ultraviolet (UV) disinfector integrated with a control system, configured to automatically deliver an effective dose of ultraviolet radiation for antibacterial and antiviral treatment of various surfaces and disinfect enclosed spaces such as hospital rooms, locker rooms, food processing facilities, and other locations where surface disinfection is desired.

### SUMMARY OF THE INNOVATION

The present invention discloses an ultraviolet (UV) disinfector integrated with a control system, configured to automatically deliver an effective dose of ultraviolet radiation for antibacterial and antiviral treatment of various surfaces.

In one embodiment, the UV disinfector is configured to automatically deliver an effective dose of ultraviolet radiation for antibacterial and antiviral treatment of various surfaces and disinfect enclosed spaces such as hospital rooms, locker rooms, food processing facilities, and other locations where surface disinfection is desired. In one embodiment, the UV disinfector is further configured to enable a user to operate in different operating modes based on the surface disinfection. In one embodiment, the UV disinfector comprises a housing having at least two sections include a top section and a base section. In one embodiment, the top section and base section are securely affixed via an anti-shock pad. In one embodiment, the housing further comprises one or more ventilation slots for allowing air to flow into and out of the housing, thereby maintaining temperature within the housing of the UV disinfector. In one embodiment, the UV disinfector further comprises a plurality of sensors, wherein the plurality of sensors is in communication of the controller configured to send one or more signals to the controller.

In one embodiment, the UV disinfector further comprises a LED indicator. The LED indicator is in communication with the controller, configured to indicate the mode of operation selected by the user. In one embodiment, the LED indicator is further configured to operate in both color and pulse-information modes. In one embodiment, the UV disinfector further comprises one or more rubberized pads. The rubberized pads are securely affixed to both sides of the top section of the housing. In one embodiment, the UV disinfector further comprises a mode key, wherein the mode key in communication with the controller is configured to enable the user to select different operating modes. In one embodiment, the mode key could be an ergonomic operating mode control key.

In one embodiment, the UV disinfector further comprises a controller having a processor and a memory in communication with the processor. In one embodiment, the memory is configured to store a set of instructions, which are executed by the processor. In one embodiment, the UV disinfector further comprises an UV light-emitting diode (LED) array. In one embodiment, the UV LED array is securely affixed to the base section of the housing. The UV LED array in communication with the controller is configured to provide a disinfecting illumination with an ultraviolet radiation to the exterior surface. In one embodiment, the UV disinfector further comprises a battery assembly. The battery assembly in communication with the controller is configured to supply electrical power to the UV LED array and the plurality of sensors. In one embodiment, a charging port is affixed to at least any one side of the housing for enabling the user to recharge the battery or batteries.

In one embodiment, the UV disinfector further comprises a reflector. The reflector is securely affixed to the base section in order to increase the utilization ratio of ultraviolet radiation emitted from the UV LED array. In one embodiment, the reflector is made of, but not limited to, stainless steel. In one embodiment, the UV disinfector further comprises a proximity sensor, charging contacts, neodymium magnets, and slip pads. In one embodiment, the neodymium magnets are affixed to the bottom portion of the housing for additional attachments.

In one embodiment, the UV disinfector is configured to perform one or more functions by the processor. The UV disinfector is configured to protect against switching on the UV LED array of in the absence of the surface being treated opposite the radiation port. In one embodiment, the UV disinfector is further configured to automatically deliver an effective dosage of ultraviolet radiation for antibacterial and antiviral treatment of various surfaces by sending control signals to the operator, thereby optimizing the UV disinfector's speed of movement. The UV disinfector could automatically provide the dosage of ultraviolet radiation based on signals received from the plurality of sensors such as, but not limited to, proximity and motion speed sensors, as well as position in space sensor, an acceleration sensor, a gyro sensor, and a temperature sensor. Based on the signals from these sensors, the built-in device controller provides the following range of smart device functions. The UV disinfector optimizes the disinfection process in terms of speed based on a selected mode of treatment depth. Automatic transition of an idle device to standby mode. The UV disinfector could exceptionally protect against the effects of hard UV radiation on the skin surface and eye's retina of the user. In one embodiment, the UV disinfector is further configured to generate a control file containing fiscal statistical information about the surface treatment carried out for a certain period, compliance with the processing speed and other operating modes of the UV disinfector. The present invention implies an interactive response of the UV disinfector allowing the adaptation of the surface treatment speed according to one of the preset dosage modes.

The use of a specialized controller of the UV disinfector creates an intelligent automated processing mode based on the readings of the plurality of sensors such as the proximity sensor, acceleration sensor, gyro sensor, and the temperature sensor. Along with the data about the exposures necessary for different types of viruses and bacteria, contained in the software of the UV disinfector, it provides a controlled disinfection mode with a guaranteed result and the ability to control the validity of the procedure.

In addition, the UV disinfector could be provided additional operational capabilities. In one embodiment, the UV disinfector is configured to enable quick installation of additional nozzles for disinfection of pipes, railings, and other volumetric surfaces. In one embodiment, the UV disinfector is further configured to enable quick installation of additional attachments for static long-term disinfection of household items (for example, cutlery, glasses, masks, hygiene items, etc.). This nozzle provides the deepest treatment with increased productivity requirements. In some embodiments, the UV disinfector is further configured to enable quick installation of an additional attachment for surface treatment with simultaneous connection of a specialized vacuum cleaner.

In one embodiment, the dosage of UV radiation and its wavelength imply professional use of the utility model UV disinfector. In one embodiment, the UV disinfector comprises at least two ports for recharging the built-in battery. The first is for a typical power connector. The second is for the contactor. The second charging method is used for automatic recharging at the moment of installation of the UV disinfector on a dock station, or a central operator unit. In one embodiment, the UV disinfector is further configured to communicate with a user's mobile device via a Bluetooth® interface. The channel is used for advanced setting of operating modes, updating firmware, reading log files.

In one embodiment, the UV disinfector could be used to disinfect small objects or daily use items such as tableware, dishes, various tools, and household items. In one embodiment, the UV disinfector further could be used to disinfect objects which are flat and transparent to optic wave of the UV range with a homogeneous surface, for example, glass, tabletops, and partitions made of glass, acrylic, polycarbonate, etc. In one embodiment, the UV disinfector further could be used to disinfect toroidal surfaces such as steering wheels of vehicles. In one embodiment, the UV disinfector further could be used to disinfect flat objects with non-uniform surfaces. In one embodiment, the UV disinfector further could be used to disinfect cylindrical objects such as pipes, railings, fencing elements, etc.

Other objects, features and advantages of the present innovation will become apparent from the following detailed description. It should be understood, however, that the detailed description and the specific examples, while indicating specific embodiments of the innovation, are given by way of illustration only, since various changes and modifications within the spirit and scope of the innovation will become apparent to those skilled in the art from this detailed description.

### BRIEF DESCRIPTION OF DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates a top perspective view of an ultraviolet (UV) disinfector, according to an embodiment of the present invention.

FIG. 2 exemplarily illustrates a bottom perspective view of the ultraviolet disinfector, according to one embodiment of the present invention.

FIG. 3 exemplarily illustrates a front view of the ultraviolet disinfector, according to one embodiment of the present invention.

FIG. 4 exemplarily illustrates a right side view of the ultraviolet disinfector, according to one embodiment of the present invention.

FIG. 5 exemplarily illustrates a left side view of the ultraviolet disinfector, according to one embodiment of the present invention.

FIG. 6 exemplarily illustrates a top view of the ultraviolet disinfector, according to one embodiment of the present invention.

FIG. 7 exemplarily illustrates a bottom view of the ultraviolet disinfector, according to one embodiment of the present invention.

FIG. 8 exemplarily illustrates a rear side view of the ultraviolet disinfector, according to one embodiment of the present invention.

FIG. 9 exemplarily illustrates an exploded view of the ultraviolet disinfector, according to one embodiment of the present invention.

FIGS. 10-16 exemplarily illustrate different views of a disinfecting apparatus for disinfecting small objects, according to another embodiment of the present invention.

FIGS. 17-18 exemplarily illustrate an external view of the disinfecting apparatus for disinfecting small objects, according to another embodiment of the present invention.

FIG. 19 exemplarily illustrates an exploded view of the disinfecting apparatus for disinfecting small objects, according to another embodiment of the present invention.

FIGS. 20-26 exemplarily illustrate different views of a disinfecting apparatus for disinfecting flat surfaces, according to another embodiment of the present invention.

FIG. 27 exemplarily illustrates an external view of the disinfecting apparatus for disinfecting flat surfaces, according to another embodiment of the present invention.

FIG. 28 exemplarily illustrates an exploded view of the disinfecting apparatus for disinfecting flat surfaces, according to another embodiment of the present invention.

FIGS. 29-35 exemplarily illustrate different views of the disinfecting apparatus for disinfecting toroidal surfaces, according to another embodiment of the present invention.

FIG. 36 exemplarily illustrates an external view of the disinfecting apparatus for disinfecting toroidal surfaces, according to another embodiment of the present invention.

FIG. 37 exemplarily illustrates an exploded view of the disinfecting apparatus for disinfecting toroidal surfaces, according to another embodiment of the present invention.

FIGS. 38-44 exemplarily illustrate different views of a disinfecting apparatus for disinfecting flat objects, according to another embodiment of the present invention.

FIG. 45 exemplarily illustrates an external view of the disinfecting apparatus for disinfecting flat objects, according to another embodiment of the present invention.

FIG. 46 exemplarily illustrates an exploded view of the disinfecting apparatus for disinfecting flat objects, according to another embodiment of the present invention.

FIGS. 47-53 exemplarily illustrate different views of a disinfecting apparatus for disinfecting cylindrical objects, according to another embodiment of the present invention.

FIG. 54 exemplarily illustrates an external view of the disinfecting apparatus for disinfecting cylindrical objects, according to another embodiment of the present invention.

FIG. 55 exemplarily illustrates an exploded view of the disinfecting apparatus for disinfecting cylindrical objects, according to another embodiment of the present invention.

FIGS. 56-61 exemplarily illustrate different views of a charging station of the UV disinfector, according to one embodiment of the present invention.

FIGS. 62-64 exemplarily illustrate external views of the charging station of the UV disinfector, according to one embodiment of the present invention.

FIG. 65 exemplarily illustrates an exploded view of the charging station of the UV disinfector, according to one embodiment of the present invention.

### DETAILED DESCRIPTION OF EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

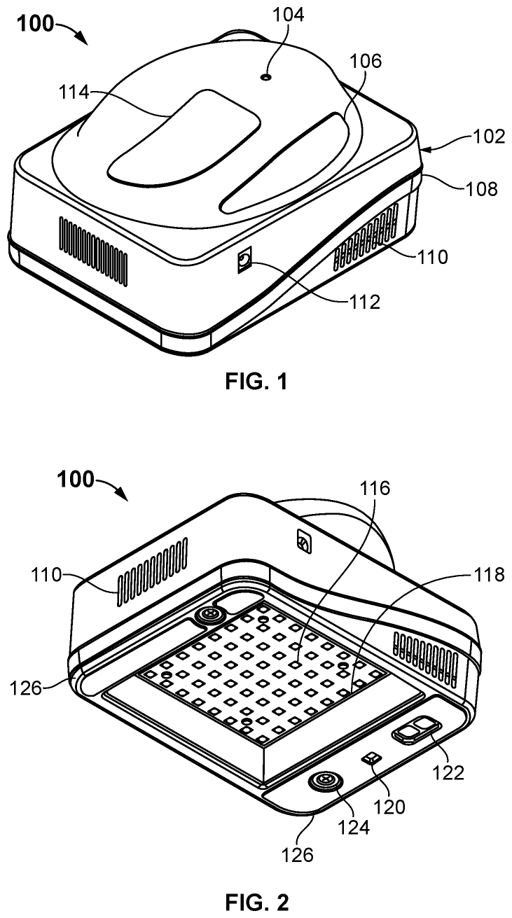

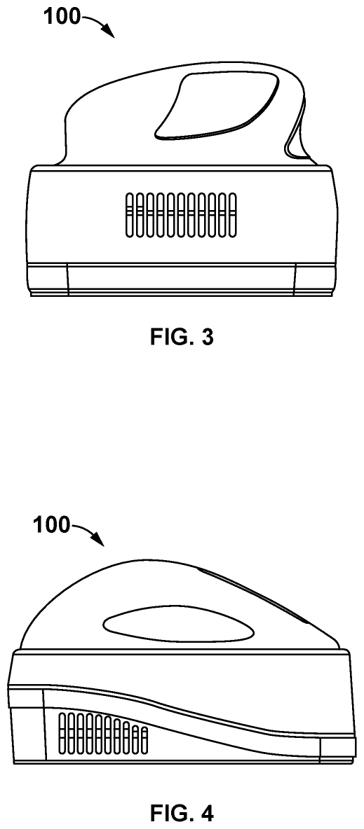

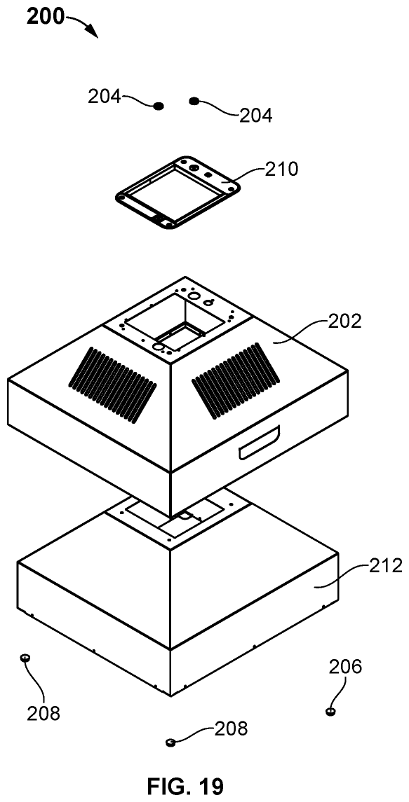

Referring to FIGS. 1-2, an ultraviolet (UV) disinfector 100 for disinfecting an exterior surface in one embodiment of the present invention is disclosed. In one embodiment, the UV disinfector 100 is configured to automatically deliver an effective dose of ultraviolet radiation for antibacterial and antiviral treatment of various surfaces and disinfect enclosed spaces such as hospital rooms, locker rooms, food processing facilities, and other locations where surface disinfection is desired. In one embodiment, the UV disinfector 100 is further configured to enable a user to operate in different operating modes based on the surface disinfection. In one embodiment, the UV disinfector 100 comprises a housing 102 having at least two sections include a top section 103 (shown in FIG. 9) and a base section 156 (shown in FIG. 9). In one embodiment, the top section 103 and base section 156 are securely affixed via an anti-shock pad 108. In one embodiment, the housing 102 further comprises one or more ventilation slots 110 for allowing air to flow into and out of the housing 102, thereby maintaining temperature within the housing 102 of the UV disinfector 100. In one embodiment, the UV disinfector 100 further comprises a plurality of sensors, wherein the plurality of sensors is in communication of the controller configured to send one or more signals to the controller, thereby controlling the operation of the UV disinfector 100. In one embodiment, the UV disinfector 100 is configured to emit ultraviolet radiation having a wavelength of about 270 nm.

In one embodiment, the UV disinfector 100 further comprises a LED indicator 104. The LED indicator 104 is in communication with the controller, configured to indicate the mode of operation selected by the user. In one embodiment, the LED indicator 104 is further configured to operate in both color and pulse-information modes. In one embodiment, the UV disinfector 100 further comprises one or more rubberized pads 106. The rubberized pads 106 are securely affixed to both sides of the top section 103 of the housing 102. In one embodiment, the UV disinfector 100 further comprises a mode key 114, wherein the mode key 114 in communication with the controller is configured to enable the user to select different operating modes. In one embodiment, the mode key 114 could be an ergonomic operating mode control key.

In one embodiment, the UV disinfector 100 further comprises a controller having a processor and a memory in communication with the processor. In one embodiment, the memory is configured to store a set of instructions, which are executed by the processor. In one embodiment, the UV disinfector 100 further comprises an UV light-emitting diode (LED) array 116. In one embodiment, the UV LED array 116 comprises at least, but not limited to, 64 LEDs. In one embodiment, the UV LED array 116 is securely affixed to the base section 156 of the housing 102. The UV LED array 116 in communication with the controller is configured to provide a disinfecting illumination with an ultraviolet radiation to the exterior surface. In one embodiment, the UV disinfector 100 further comprises a battery assembly 134 (shown in FIG. 9). The battery assembly 134 in communication with the controller is configured to supply electrical power to the UV LED array 116 and the plurality of sensors. In one embodiment, a charging port 112 is affixed to at least any one side of the housing 102 for enabling the user to recharge the battery or batteries.

In one embodiment, the UV disinfector 100 further comprises a reflector 118. The reflector 118 is securely affixed to the base section 156 in order to increase the utilization ratio of ultraviolet radiation emitted from the UV LED array 116. In one embodiment, the reflector 118 is made of, but not limited to, stainless steel. In one embodiment, the UV disinfector 100 further comprises a proximity sensor 120, charging contacts 122, neodymium magnets 124, and slip pads 126. In one embodiment, the neodymium magnets 124 are affixed to the bottom portion of the housing 102 for additional attachments. In one embodiment, the range of the proximity sensor 120 is about 1-20 mm (adjustable in software).

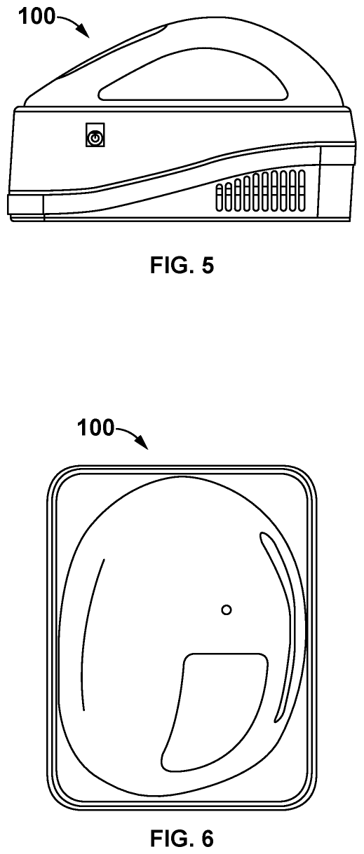

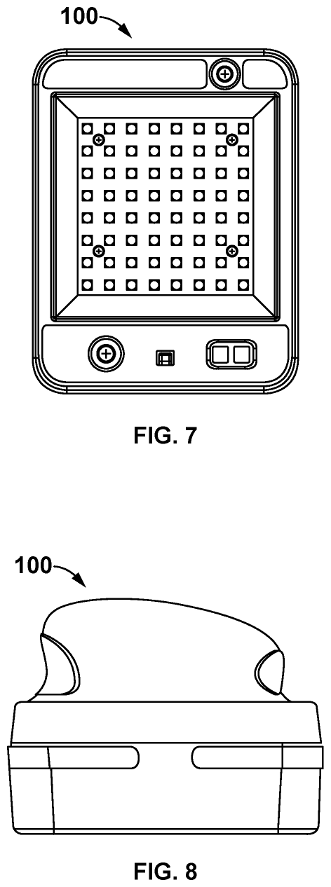

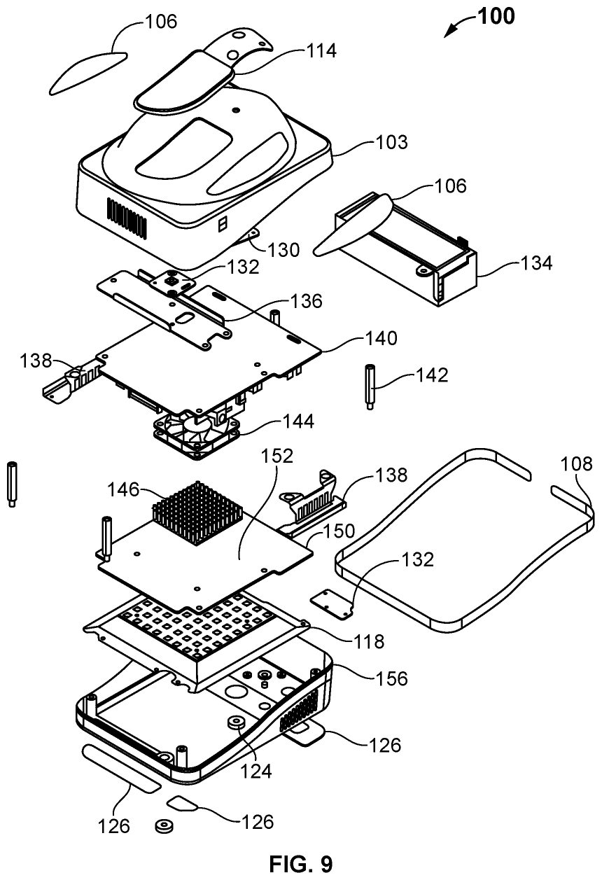

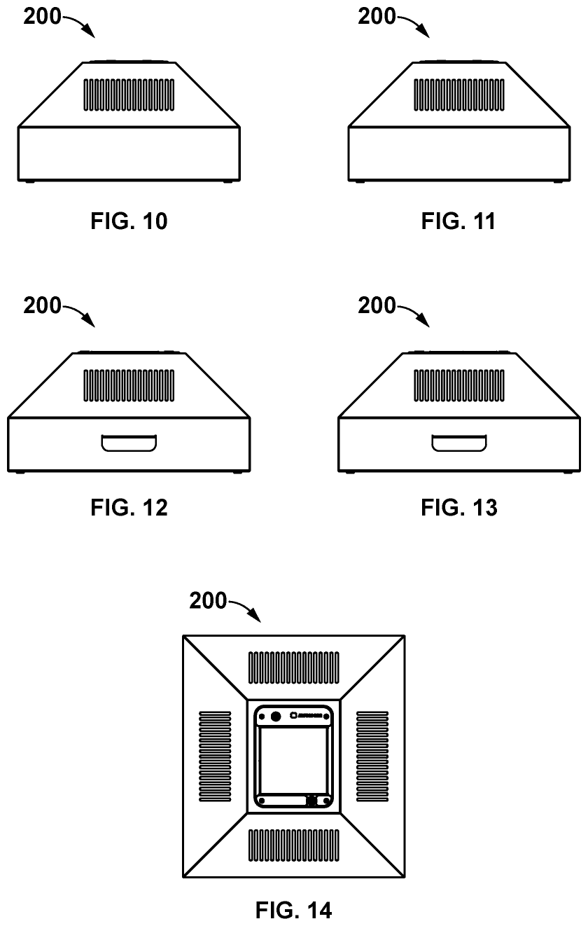

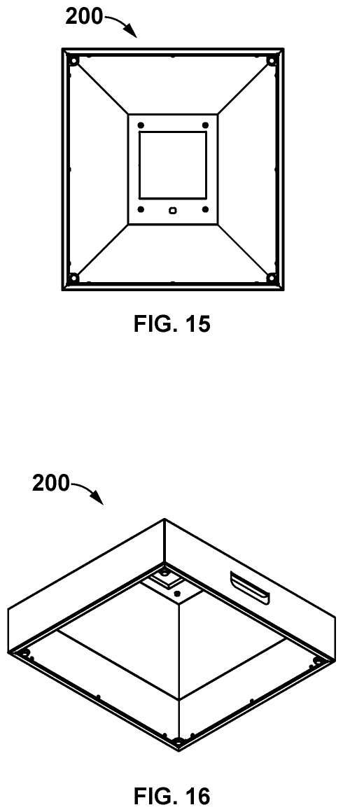

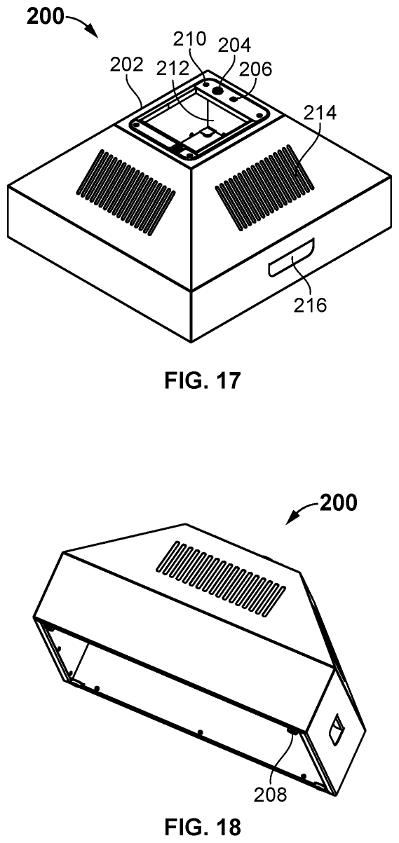

Referring to FIGS. 3-8, different views of the UV disinfector 100 are disclosed. In one embodiment, the UV disinfector 100 is configured to perform one or more functions by the processor. The UV disinfector 100 is configured to protect against switching on the UV LED array 116 (shown in FIG. 2) in the absence of the surface being treated opposite the radiation port. In one embodiment, the UV disinfector 100 is further configured to automatically deliver an effective dosage of ultraviolet radiation for antibacterial and antiviral treatment of various surfaces by sending control signals to the operator, thereby optimizing the UV disinfector's speed of movement. The UV disinfector 100 could automatically provide the dosage of ultraviolet radiation based on signals received from the plurality of sensors such as, but not limited to, proximity and motion speed sensors, as well as position in space sensor, an acceleration sensor, a gyro sensor, and a temperature sensor. Based on the signals from these sensors, the built-in device controller provides the following range of smart device functions. The UV disinfector 100 optimizes the disinfection process in terms of speed based on a selected mode of treatment depth. Automatic transition of an idle device to standby mode. The UV disinfector 100 could provide exceptional protection against the effects of hard UV radiation on the skin surface and eye's retina of the user. In one embodiment, the UV disinfector 100 is further configured to generate a control file containing fiscal statistical information about the surface treatment carried out for a certain period, compliance with the processing speed, and other operating modes of the UV disinfector 100. The present invention implies an interactive response of the UV disinfector 100 allowing the adaptation of the surface treatment speed according to one of the preset dosage modes.

The use of a specialized controller of the UV disinfector 100 creates an intelligent automated processing mode based on the readings of the plurality of sensors such as the proximity sensor, acceleration sensor, gyro sensor, and temperature sensor. Along with the data about the exposures necessary for different types of viruses and bacteria, contained in the software of the UV disinfector 100, it provides a controlled disinfection mode with a guaranteed result and the ability to control the validity of the procedure.

In addition, the UV disinfector 100 could be provided additional operational capabilities. In one embodiment, the UV disinfector 100 is configured to enable quick installation of additional nozzles for disinfection of pipes, railings, and other volumetric surfaces. In one embodiment, the UV disinfector 100 is further configured to enable quick installation of additional attachments for static long-term disinfection of household items (for example, cutlery, glasses, masks, hygiene items, etc.). This nozzle provides the deepest treatment with increased productivity requirements. In some embodiments, the UV disinfector 100 is further configured to enable quick installation of an additional attachment for surface treatment with simultaneous connection of a specialized vacuum cleaner. In one embodiment, the UV disinfector 100 further comprises a magnetic mount 124, which is integrated into the device for the installation of additional equipment.

In one embodiment, the dosage of UV radiation and its wavelength implies professional use of the utility model UV disinfector 100. In one embodiment, the UV disinfector 100 comprises at least two ports for recharging the built-in battery. The first is for a typical power connector. The second is for the contactor. The second charging method is used for automatic recharging at the moment of installation of the UV disinfector 100 on a dock station, or a central operator unit. In one embodiment, the UV disinfector 100 is further configured to communicate with a user's mobile device via a Bluetooth® interface. The channel is used for the advanced setting of operating modes, updating firmware, reading log files. In one embodiment, the UV disinfector 100 has optical radiation power of about 1150 mW. In one embodiment, the UV disinfector 100 could treat the surface about (static) 0.01 m2. In one embodiment, the UV disinfector 100 could require a charger of 19 V. In one embodiment, the built-in battery has the capacity of about 3600 mA/h and the voltage of the built-in battery is about 18.8 V. In one embodiment, the UV disinfector 100 has the dimensions of about, but not limited to, 120×154×92 mm and the weight is about, but not limited to, 450 g.

Referring to FIG. 9, an exploded view of the UV disinfector 100 in one embodiment is disclosed. In one embodiment, the top section 103 and base section 156 are configured to securely fasten together using one or more fasteners 142, for example, distancing brass stands via a shock-resistant plastic buffer 108. The rubberized pads 106 are securely affixed to both sides of the top section 103 of the housing 102. In one embodiment, an enclosure component key or mode key 114 is affixed to the top section of the UV disinfector 100.

In one embodiment, the UV disinfector 100 further comprises a printed circuit board (PCB) 130 for the LED indicator 104 and a button PCB 132, which is mounted on a bracket 136. In one embodiment, the battery assembly 134 is configured to supply electrical power to the controller, UV LED array 116, and the plurality of sensors. In one embodiment, the UV disinfector 100 further comprises a cooler unit 144, which is securely mounted on cooler unit holders 138. In one embodiment, the controller of the UV disinfector 100 could be securely connected to the controller PCB 140. In one embodiment, the proximity sensor 120 is connected to the Proximity sensor PCB 152. In one embodiment, a spiked heat sink AL146 is securely connected to the UV LED PCB 150 for providing optimal cooling.

In one embodiment, the UV disinfector 100 could be provided with a radiator, a PCB based on aluminum alloy, and forced active ventilation for maintaining optimal temperature parameters for the operation of UV LED array 116. The absence of gas-discharge components in the design of the UV disinfector 100 makes it possible to operate the device in a repeated-short-term mode, which implies instant readiness of the device for operation immediately after the previous shutdown.

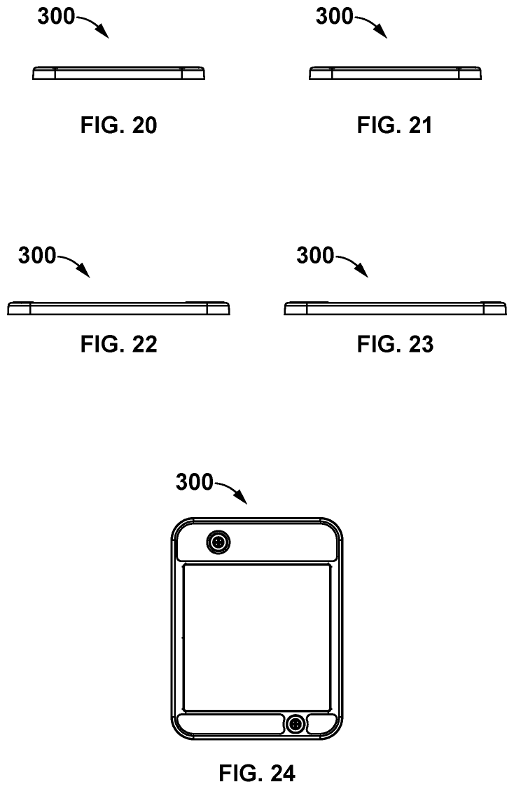

Referring to FIGS. 10-16, different views of a disinfecting apparatus 200 configured to attach to the UV disinfector 100 for disinfecting small objects, according to another embodiment of the present invention. In one embodiment, the apparatus 200 is attached to the UV disinfector 100 that allows quick and comfortable UV disinfection of small objects. The apparatus 200 contains design solutions and hardware ports that provide improved functionality in conjunction with the UV disinfector 100. In one embodiment, the apparatus 200 allows quick and comfortable disinfection of small objects. In one embodiment, the apparatus 200 is used for disinfecting daily use items such as tableware, dishes, various tools, and household items. The apparatus 200 disinfects small objects/items as efficiently and safely as possible for others.

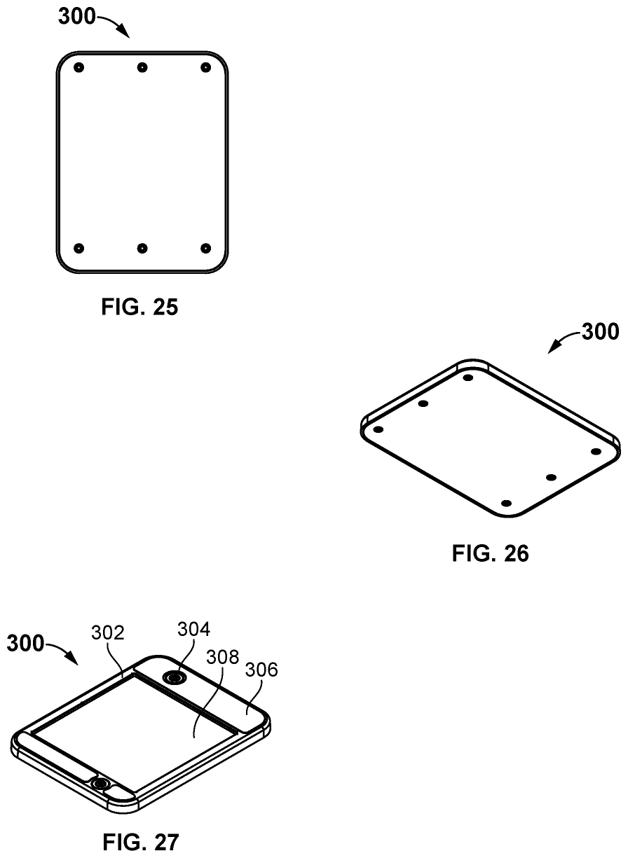

Referring to FIGS. 17-18, different external views of the disinfecting apparatus 200 for disinfecting small objects, according to another embodiment of the present invention. In one embodiment, the apparatus 200 comprises a metal outer casing or metal housing or enclosure 202. In one embodiment, the apparatus 200 further comprises one or more neodymium magnets 204 configured to provide quick installation and removal from the UV disinfector 100. In one embodiment, the apparatus 200 further comprises one or more proximity sensor windows 206 configured to sense the exterior surface for disinfecting each tool. In one embodiment, the apparatus 200 further comprises a UV reflector 212 for reflecting UV light on the surface of the object. In one embodiment, the metal outer casing 202 is equipped with a plurality of ventilation holes/slots 214 for allowing the air to flow in and out of the metal outer casing 202, thereby maintaining the temperature within the metal outer casing 202.

In one embodiment, the apparatus 200 further comprises at least a pair of carrying handles 216 on the sides of the metal outer casing 202. In one embodiment, the metal outer casing 202 is equipped with handles 216 for carrying and easy installation on the object being treated. In one embodiment, the apparatus 200 is provided with a plastic plat band 210 at the top of the metal outer casing 202. In one embodiment, the apparatus 200 further comprises one or more rubberized pads 208. The rubberized pads 208 are securely affixed at the bottom corners of the metal outer casing 202.

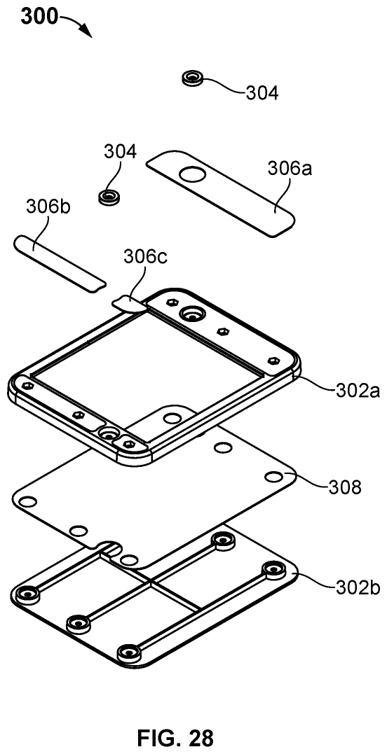

Referring to FIG. 19, an exploded view of the internal components of the disinfecting apparatus 200 for disinfecting small objects, according to another embodiment of the present invention. The apparatus 200 has a dimension of about 360×330×181 mm. The apparatus 200 has a maximum treated surface having a dimension of about 310×280 mm. The apparatus 200 has a weight of about 2537 g. In one embodiment, the apparatus 200 comprises a metal outer casing or metal housing or enclosure 202. The metal outer casing 202 is configured to enclose a plurality of internal components. In one embodiment, the metal outer casing 202 is provided with a pair of magnets 204 and one or more sensors 206 at the top of a plastic plat band 210. In one embodiment, the metal outer casing 202 comprises at least two magnets 204. In one embodiment, the magnets 204 are neodymium magnets having a dimension of about 12×3×7-3.5. In one embodiment, the magnets 204 are provided for quick installation and removal of the apparatus 200 from the UV disinfector 100. In one embodiment, the sensors 206 are proximity sensor windows. The apparatus 200 prevents turning on sensors 206 for the presence of the treated surface while working in conjunction with the UV disinfector 100.

In one embodiment, the apparatus 200 further comprises a cavity at its lower portion for the placement of a UV reflector 212 for reflecting UV light on the surface of the object. In one embodiment, the UV reflector 212 is a polished stainless steel deflector to maximize treatment efficiency. In one embodiment, the apparatus 200 is further equipped with a plurality of ventilation holes 214 on the outer surface of the metal outer casing 202 to prevent the formation of surface condensation on the treated objects. In one embodiment, the metal outer casing 202 is provided with the carrying handles 216 on opposite sides for easy handling and installation on the object being treated. In one embodiment, the carry handle 216 at the sides support easy handling of the disinfector while disinfecting objects such as tableware and other tools. In one embodiment, the apparatus 200 is further equipped with one or more rubberized pads or rubber stand 208. In one embodiment, the apparatus 200 is equipped with at least four rubberized pads 208 that allow the apparatus 200 to operate on inclined surfaces and preventing them from scratching or other damage. Further, the apparatus 200 attached to the UV disinfector 100 could be connected to the charger.

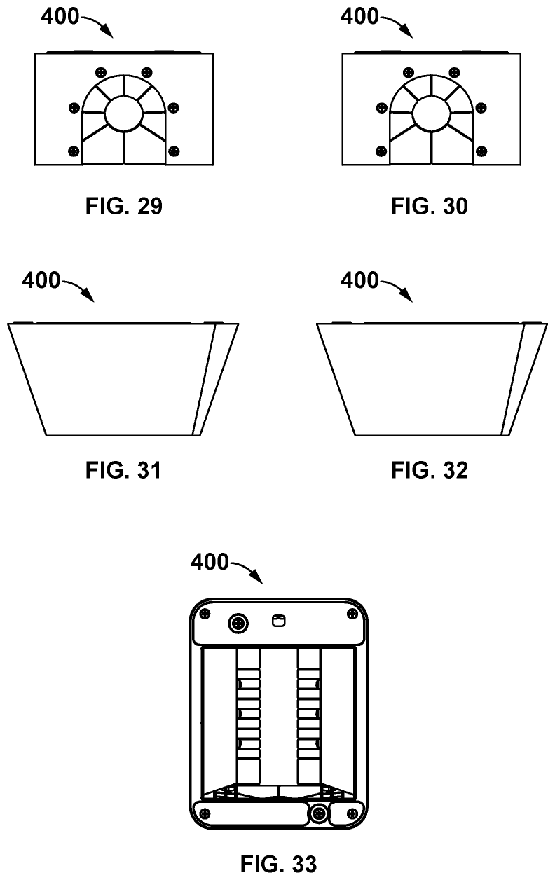

Referring to FIGS. 20-26, different views of a disinfecting apparatus 300 configured to attach to the UV disinfector 100 for disinfecting flat surfaces, according to another embodiment of the present invention. In one embodiment, the apparatus 300 is configured to attach to the UV disinfector 100 that allows quick and comfortable UV disinfection of flat surfaces. The apparatus 300 contains design solutions and hardware ports that provide improved functionality in conjunction with the UV disinfector 100. In one embodiment, the apparatus 300 is used for disinfecting objects which are flat and transparent to optic wave of the UV range with a homogeneous surface, for example, glass, tabletops, and partitions made of glass, acrylic, polycarbonate, etc.

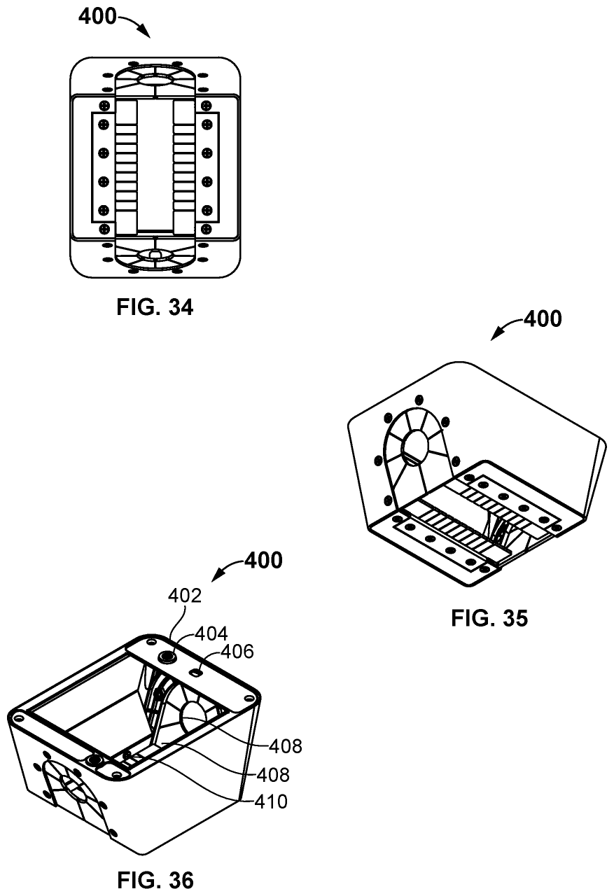

Referring to FIG. 27, an external view of the disinfecting apparatus 300 for disinfecting flat surfaces, according to another embodiment of the present invention. In one embodiment, the apparatus 300 comprises a plastic enclosure 302 configured to enclose a plurality of internal components. In one embodiment, the apparatus 300 further comprises one or more magnets 304. In one embodiment, the magnets 304 are neodymium magnets. In one embodiment, the apparatus 300 further comprises one or more polytetrafluoroethylene (PTFE) slip plates 306. The PTFE slip plates 306 act as a non-wetting surface and resistant to high temperatures. In one embodiment, the apparatus 300 further comprises a UV reflector screen 308 that delivers the UV light for disinfecting the flat surface.

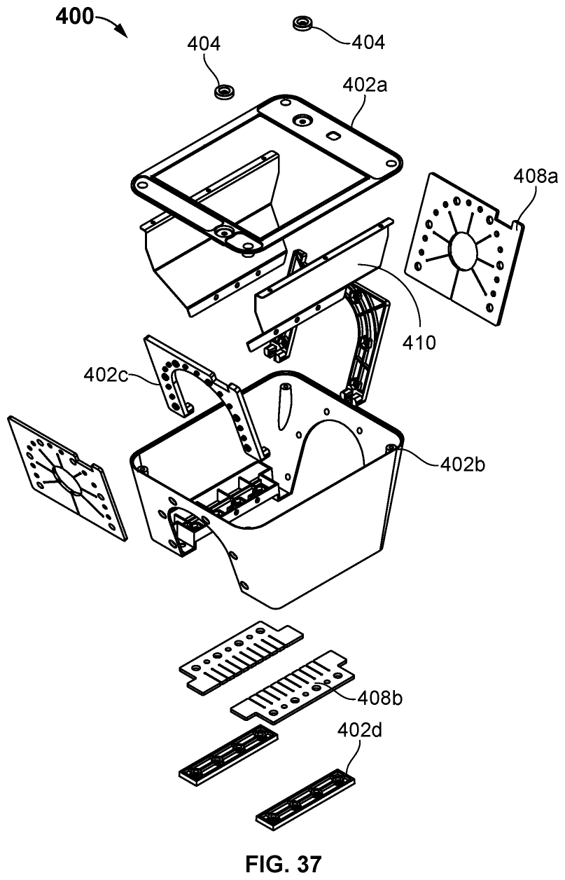

Referring to FIG. 28, an exploded view of the disinfecting apparatus 300 for disinfecting flat surfaces, according to another embodiment of the present invention. The apparatus 300 has a dimension of about 120×154×9 mm. The apparatus 300 has a maximum thickness of the treated material having the size of about 20 mm. The apparatus 300 has a weight of about 79 g. In one embodiment, the apparatus 300 comprises a top section having a plastic enclose 302. The plastic enclosure 302 has at least two parts include a first enclosure part 302a and a second enclosure part 302b. In one embodiment, the apparatus 300 further comprises one or more magnets 304 affixed to the top section. In one embodiment, the magnets 304 are neodymium magnets. In one embodiment, the apparatus 300 comprises at least two magnets 304 having the dimension of about 12×3×7-3.5.

In one embodiment, the apparatus 300 further comprises one or more polytetrafluoroethylene (PTFE) slip plates 306 and a UV reflector screen 308. In one embodiment, the one or more PTFE slip plates 306 includes a first PTFE sliding gasket 306a, a second PTFE sliding gasket 306b, and a third PTFE sliding gasket 306c. In one embodiment, the UV reflector screen 308 is a polished stainless steel deflector to maximize treatment efficiency. In one embodiment, the UV reflector screen 308 is provided between the first enclosure part 302a and the second enclosure part 302b. Further, the enclosure parts (302a, 302b), PTFE slip plates (306a, 306b, 306c), and UV reflector screen 308 are fastened together using one or more fasteners. Further, the apparatus 300 supports the operation of the sensor of the presence of the treated surface when working in conjunction with the disinfector.

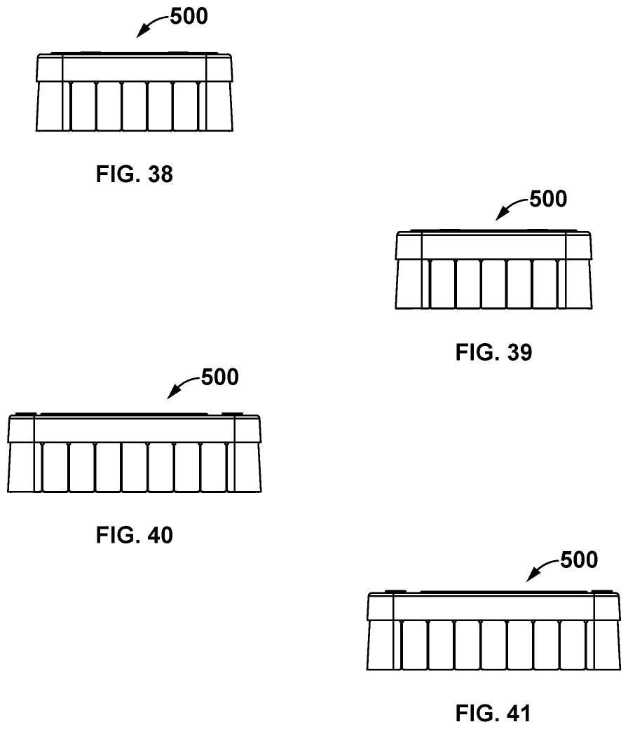

Referring to FIGS. 29-35, different views of a disinfecting apparatus 400 configured to attach to the UV disinfector 100 for disinfecting toroidal surfaces, according to another embodiment of the present invention. In one embodiment, the apparatus 400 is attached to the UV disinfector 100 that allows quick and comfortable UV disinfection of toroidal surfaces. The apparatus 400 contains design solutions and hardware ports that provide improved functionality in conjunction with the UV disinfector 100. In one embodiment, the apparatus 400 is used for disinfection of toroidal surfaces for example, steering wheels of vehicles.

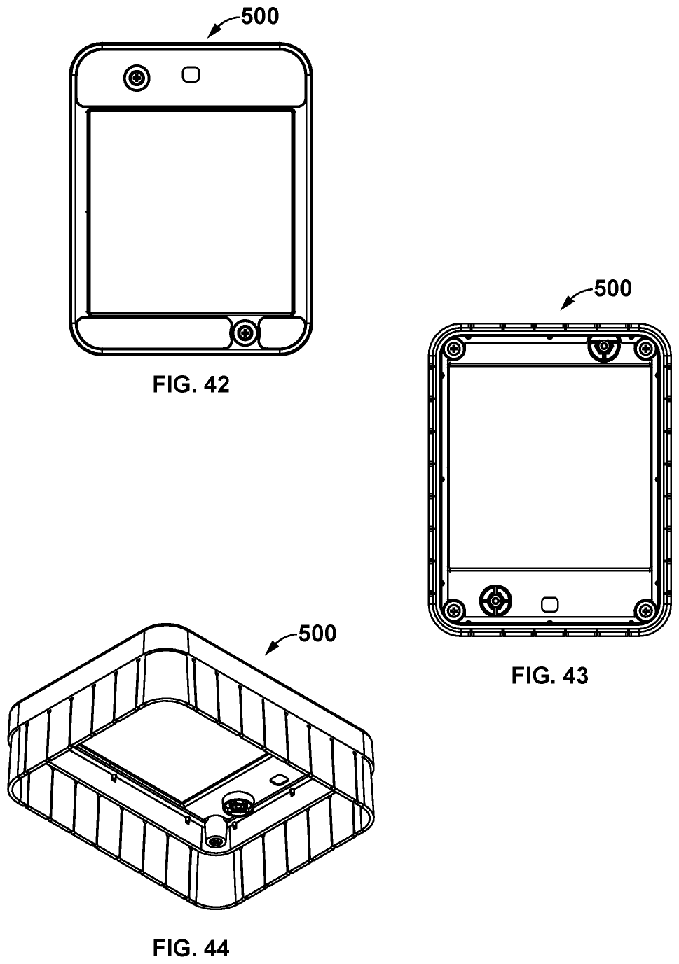

Referring to FIG. 36, an external view of the disinfecting apparatus 400 for disinfecting toroidal surfaces, according to another embodiment of the present invention. In one embodiment, the apparatus 400 comprises a plastic enclosure 402 configured to enclose a plurality of internal components. In one embodiment, the apparatus 400 further comprises one or more magnets 404. In one embodiment, the magnets 404 are neodymium magnets. In one embodiment, the apparatus 400 further comprises one or more sensors 406. In one embodiment, the sensors 406 are proximity sensor windows. In one embodiment, the apparatus 400 further comprises one or more UV protective rubber shutters 408 configured to prevent the escape of UV radiation from the treatment area. In one embodiment, the apparatus 400 further comprises a UV reflector 410. In one embodiment, the UV reflector 410 is a stainless steel UV reflector that delivers the UV light for disinfecting the toroidal surface. At least 98% of the radiation energy is retained inside the housing during treatment.

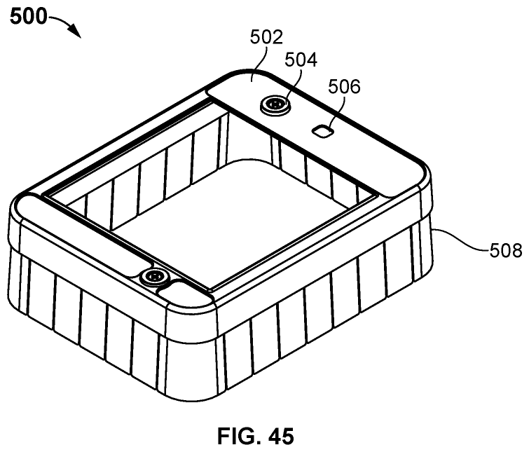

Referring to FIG. 37, an exploded view of the disinfecting apparatus 400 for disinfecting toroidal surfaces, according to another embodiment of the present invention. The apparatus 400 is attached to the UV disinfector 100 to disinfect the toroidal surfaces such as steering wheels of the vehicles as efficiently and safely as possible for others. The apparatus 400 has a dimension of about 120×154×75 mm. The apparatus 400 has a treated surface having a dimension of about 25 to 50 mm. The apparatus 400 has a toroidal section having an optimal radius of about 380 mm. The apparatus 400 has a weight of about 175 g.

In one embodiment, the apparatus 400 comprises a plastic enclosure 402 configured to enclose a plurality of internal components. In one embodiment, the plastic enclosure 402 has at least four parts include a first enclosure part 402a, a second enclosure part 402b, a third enclosure part 402c, and a fourth enclosure part 402d. The third and fourth enclosure parts (402c, 402d) have a pair of enclosure plates. In one embodiment, the apparatus 400 further comprises one or more magnets 404. In one embodiment, the magnets 404 are neodymium magnets. In one embodiment, the apparatus 400 has at least two magnets 404 having the dimension of about 12×3×7-3.5. In one embodiment, the apparatus 400 is equipped with locks based on the neodymium magnets 404, which provide the fastest possible replacement of the apparatus 400.

In one embodiment, the apparatus 400 further comprises one or more UV protective rubber shutter 408. In one embodiment, the UV protective rubber shutter 408 is an elastic protective shutter. The UV protective rubber shutter 408 is configured to prevent the escape of UV radiation from the treatment area. At least 98% of the radiation energy is retained inside the apparatus 400 during treatment. The UV protective rubber shutter 408 has at least two parts include a first UV protective rubber shuttle 408a and a second UV protective rubber shuttle 408b. Each UV protective rubber shuttle (408a, 408b) has at least two shuttle plates. In one embodiment, the apparatus 400 further comprises a UV reflector 410. In one embodiment, the apparatus 400 has at least two UV reflectors 410. In one embodiment, the UV reflector 410 is a stainless steel UV reflector that delivers the UV light for disinfecting the toroidal surface. In one embodiment, the UV reflectors 410 are made of polished stainless steel that provides 360° toroidal surface treatment in a single pass.

In one embodiment, the apparatus 400 is equipped with a hardware optical gateway that prevents UV radiation from escaping into the gap between the apparatus 400 and the UV disinfector 100. In addition, the apparatus 400 supports the operation of the sensor of the presence of the treated surface while working in conjunction with the UV disinfector 100. Further, the apparatus 400 is supplied with a firmware update for the UV disinfector 100.

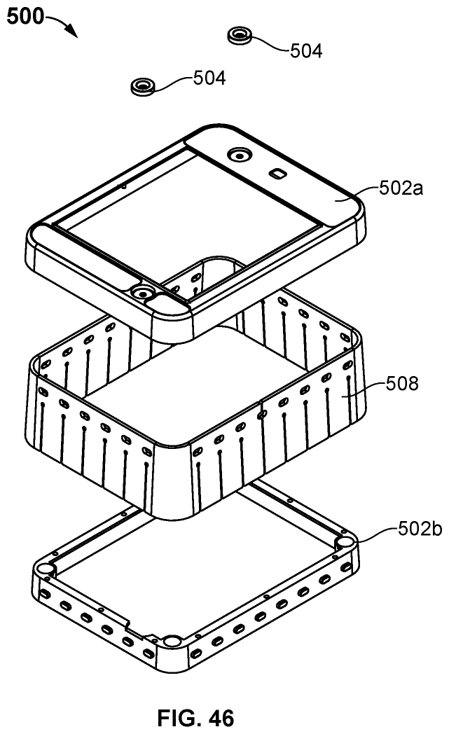

Referring to FIGS. 38-44, different views of a disinfecting apparatus 500 configured to attach to the UV disinfector 100 for disinfecting flat objects, according to another embodiment of the present invention. In one embodiment, the apparatus 500 is attached to the UV disinfector 100 that allows quick and comfortable UV disinfection of flat objects with non-uniform surfaces. The apparatus 500 contains design solutions and hardware ports that provide improved functionality in conjunction with the UV disinfector 100. In one embodiment, the apparatus 500 is attached to the UV disinfector 100 configured to disinfect the flat objects with non-uniform surfaces as efficiently and safely as possible.

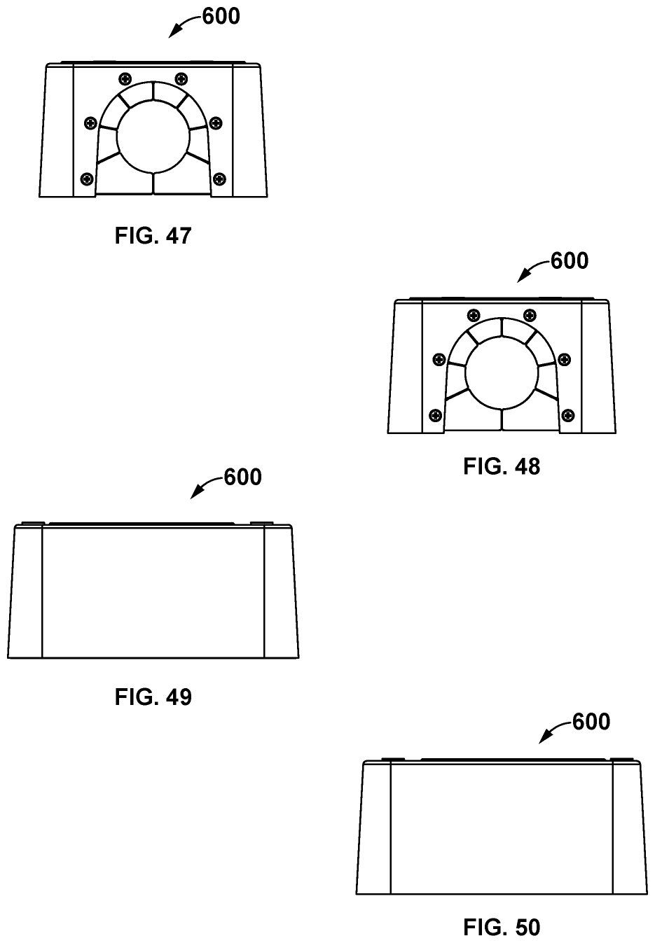

Referring to FIG. 45, an external view of the disinfecting apparatus 500 for disinfecting flat objects with non-uniform surfaces, according to another embodiment of the present invention. In one embodiment, the apparatus 500 comprises a plastic enclosure 502 configured to enclose a plurality of internal components. In one embodiment, the apparatus 500 further comprises one or more magnets 504. In one embodiment, the magnets 504 are neodymium magnets. In one embodiment, the apparatus 500 further comprises one or more sensors 506. In one embodiment, the sensors 506 are proximity sensor windows. In one embodiment, the apparatus 500 further comprises one or more UV protective rubber shutters 508 configured to prevent the escape of UV radiation from the treatment area. In one embodiment, the apparatus 400 further comprises a UV reflector 410.

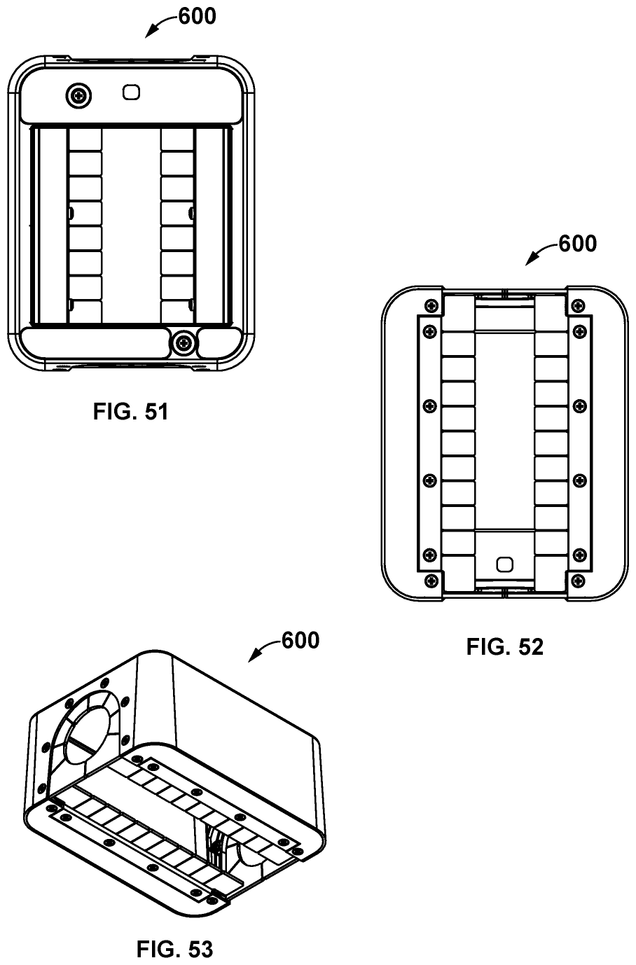

Referring to FIG. 46, an exploded view of the disinfecting apparatus 500 for disinfecting flat objects with non-uniform surfaces, according to another embodiment of the present invention. The apparatus 500 has a dimension of about 120×154×48 mm. The apparatus 500 has a treated surface heterogeneity having a dimension of about 30 mm. The apparatus 500 has a weight of about 97 g. In one embodiment, the apparatus 500 comprises a plastic enclosure 502 configured to enclose a plurality of internal components. In one embodiment, the plastic enclosure 502 has at least two parts include a first enclosure part 502a and a second enclosure part 502b. In one embodiment, the apparatus 500 further comprises one or more magnets 504. In one embodiment, the magnets 504 are neodymium magnets. In one embodiment, the apparatus 500 has at least two magnets 504 having the dimension of about 12×3×7-3.5. In one embodiment, the apparatus 500 is equipped with locks based on the neodymium magnets 504, which provide the fastest possible replacement of the apparatus 500.

In one embodiment, the apparatus 500 further comprises one or more UV protective rubber shutter 508. In one embodiment, the UV protective rubber shutter 508 is an elastic protective shutter. The UV protective rubber shutter 508 is configured to prevent the escape of UV radiation from the treatment area. At least 98% of the radiation energy is retained inside the apparatus 500 during treatment. In one embodiment, the apparatus 500 is equipped with a hardware optical gateway that prevents UV radiation from escaping into the gap between the apparatus 500 and the UV disinfector 100. In addition, the apparatus 500 supports the operation of the sensor of the presence of the treated surface while working in conjunction with the UV disinfector 100. Further, the apparatus 500 is supplied with a firmware update for the UV disinfector 100.

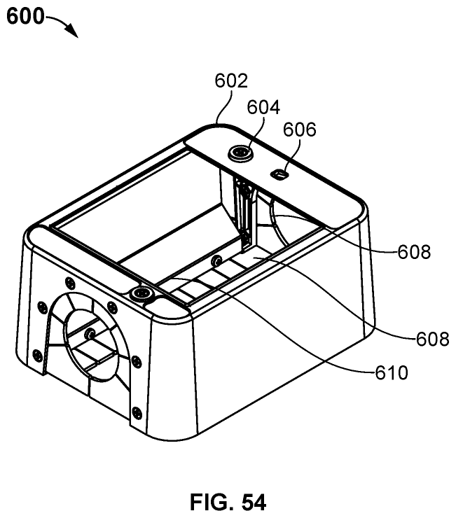

Referring to FIGS. 47-53, different views of a disinfecting apparatus 600 configured to attach to the UV disinfector 100 for disinfecting cylindrical objects, according to another embodiment of the present invention. In one embodiment, the apparatus 600 is attached to the UV disinfector 100 that allows quick and comfortable UV disinfection of cylindrical objects. The apparatus 600 contains design solutions and hardware ports that provide improved functionality in conjunction with the UV disinfector 100. In one embodiment, the apparatus 600 is used for disinfection of cylindrical objects, for example, pipes, railings, fencing elements, etc. In one embodiment, the apparatus 600 is attached to the UV disinfector 100 configured to disinfect the cylindrical objects such as pipes, railings, fencing elements, etc., including those equipped with lateral support elements with maximum efficiency.

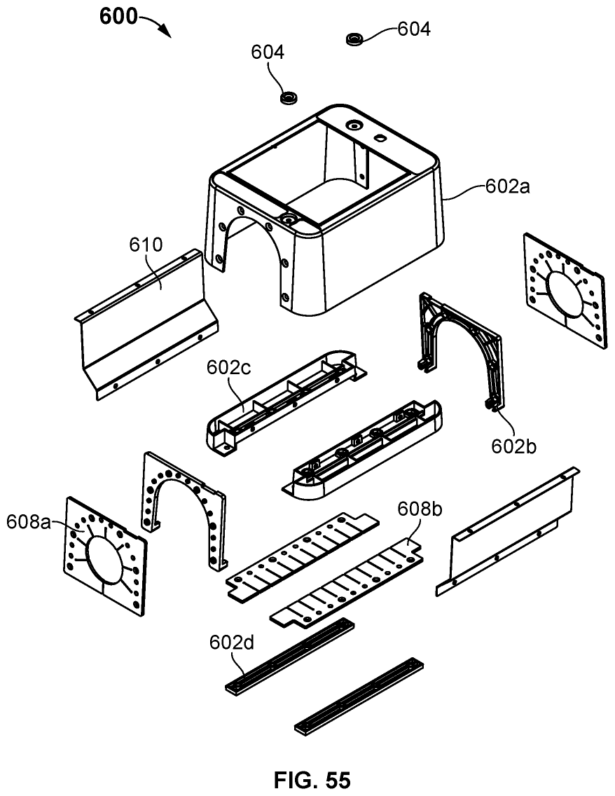

Referring to FIG. 54, an external view of the disinfecting apparatus 600 for disinfecting cylindrical objects, according to another embodiment of the present invention. In one embodiment, the apparatus 600 comprises a plastic enclosure 602 configured to enclose a plurality of internal components. In one embodiment, the apparatus 600 further comprises one or more magnets 604. In one embodiment, the magnets 604 are neodymium magnets. In one embodiment, the apparatus 600 further comprises one or more sensors 606. In one embodiment, the sensors 606 are proximity sensor windows. In one embodiment, the apparatus 600 further comprises one or more UV protective rubber shutters 608 configured to prevent the escape of UV radiation from the treatment area. In one embodiment, the apparatus 600 further comprises a UV reflector 610. In one embodiment, the UV reflector 610 is a stainless steel UV reflector that delivers the UV light for disinfecting cylindrical objects. At least 98% of the radiation energy is retained inside the housing during treatment.

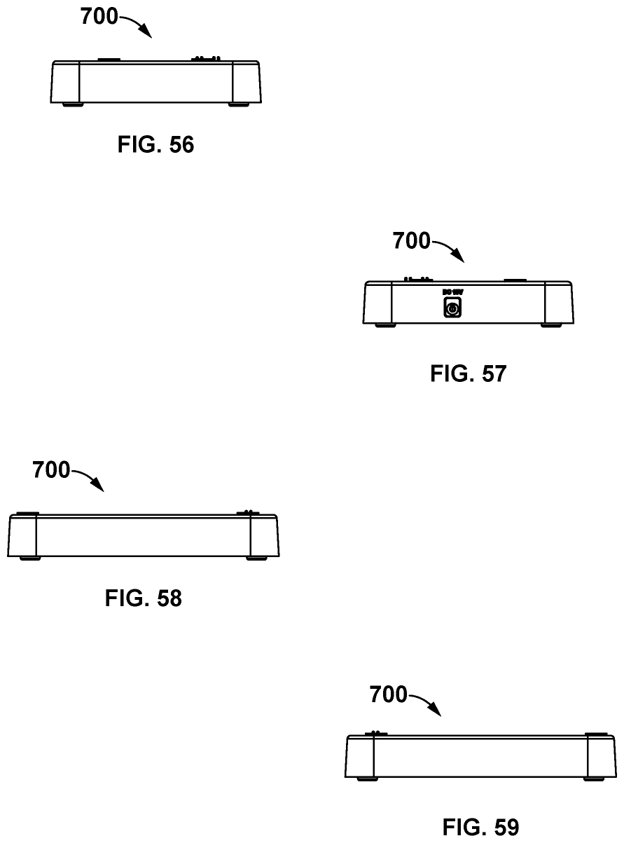

Referring to FIG. 55, an exploded view of the disinfecting apparatus 600 for disinfecting cylindrical objects, according to another embodiment of the present invention. The apparatus 600 is attached to the UV disinfector 100 to disinfect the cylindrical objects such as pipes, railings, fencing elements, etc., as efficiently and safely as possible for others. The apparatus 600 has a dimension of about 120×154×74 mm. The apparatus 600 has a maximum treated surface having a dimension of about 60 mm. The apparatus 600 has a cylindrical section having a minimum radius of about 500 mm. The apparatus 600 has a weight of about 184 g.

In one embodiment, the apparatus 600 comprises a plastic enclosure 602 configured to enclose a plurality of internal components. In one embodiment, the plastic enclosure 602 has at least four parts include a first enclosure part 602a, a second enclosure part 602b, a third enclosure part 602c, and a fourth enclosure part 602d. The second, third, and fourth enclosure parts (602b, 602c, 602d) has a pair of enclosure plates. In one embodiment, the apparatus 600 further comprises one or more magnets 604. In one embodiment, the magnets 604 are neodymium magnets. In one embodiment, the apparatus 600 has at least two magnets 604 having the dimension of about 12×3×7-3.5. In one embodiment, the apparatus 600 is equipped with locks based on the neodymium magnets 604, which provide the fastest possible replacement of the apparatus 600.

In one embodiment, the apparatus 600 further comprises one or more UV protective rubber shutter 608. In one embodiment, the UV protective rubber shutter 608 is an elastic protective shutter. The UV protective rubber shutter 608 is configured to prevent the escape of UV radiation from the treatment area. At least 98% of the radiation energy is retained inside the apparatus 600 during treatment. The UV protective rubber shutter 608 has at least two parts include a first UV protective rubber shuttle 608a and a second UV protective rubber shuttle 608b. Each UV protective rubber shuttle (608a, 608b) has at least two shuttle plates. In one embodiment, the apparatus 600 further comprises a UV reflector 610. In one embodiment, the apparatus 600 has at least two UV reflectors 610. In one embodiment, the UV reflector 610 is a stainless steel UV reflector that delivers the UV light for disinfecting cylindrical objects. In one embodiment, the UV reflectors 610 are made of polished stainless steel that provides 360° cylindrical surface treatment in a single pass.

In one embodiment, the apparatus 600 is equipped with a hardware optical gateway that prevents UV radiation from escaping into the gap between the apparatus 600 and the UV disinfector 100. In addition, the apparatus 600 supports the operation of the sensor of the presence of the treated surface while working in conjunction with the UV disinfector 100. Further, the apparatus 600 is supplied with a firmware update for the UV disinfector 100.

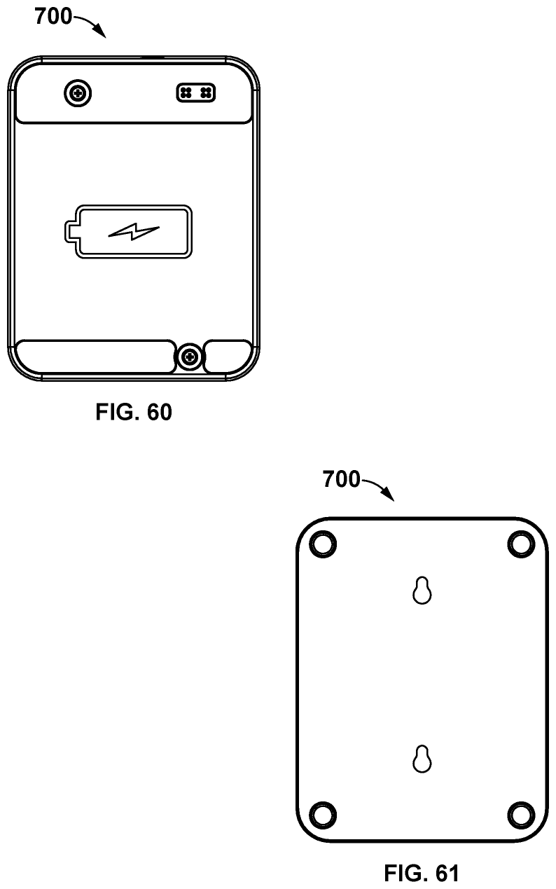

Referring to FIGS. 56-61, different views of a charging station 700 of the UV disinfector 100, according to one embodiment of the present invention. In one embodiment, the charging station 700 is attached to the UV disinfector 100 that allows quick and comfortable charging of the UV disinfector 100. The apparatus 700 contains design solutions and hardware ports that provide improved functionality in conjunction with the UV disinfector 100. In one embodiment, the apparatus 700 is attached to the UV disinfector 100 configured to prompt the recharging of the UV disinfector 100 without connecting to the power supply connector.

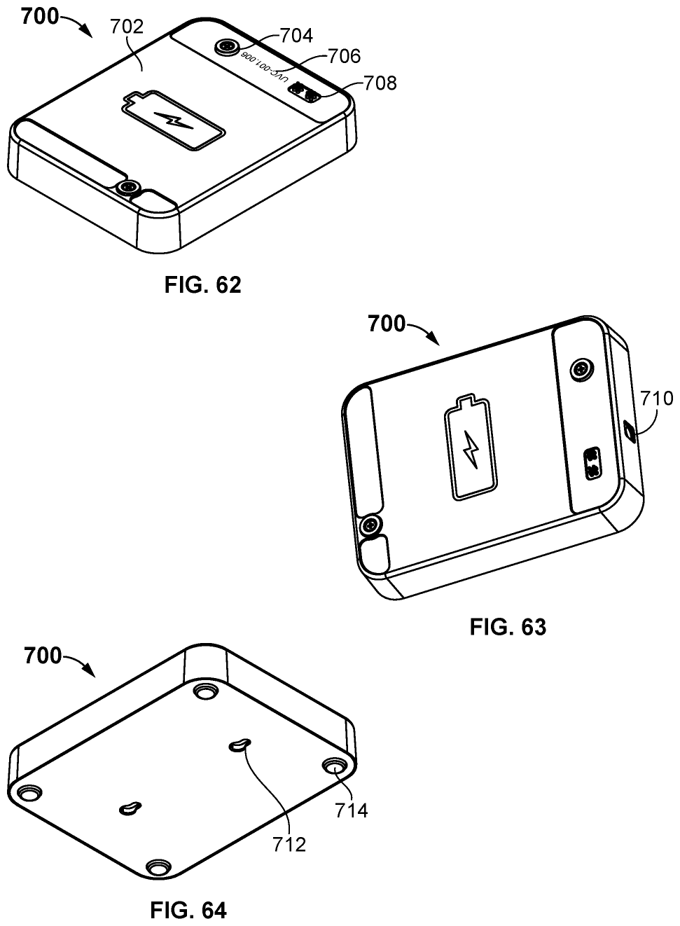

Referring to FIGS. 62-64, external views of the charging station 700 of the UV disinfector 100, according to one embodiment of the present invention. In one embodiment, the apparatus 700 comprises a plastic enclosure 702 configured to enclose a plurality of internal components. In one embodiment, the apparatus 700 further comprises one or more magnets 704. In one embodiment, the magnets 704 are neodymium magnets. In one embodiment, the apparatus 700 further comprises a device number 706 and a spring contact connector 708. In one embodiment, the apparatus 700 further comprises a power supply connector 710 provided at its upper end. In one embodiment, the apparatus 700 further comprises one or more wall mount holes 712 and one or more rubberized pads 714.

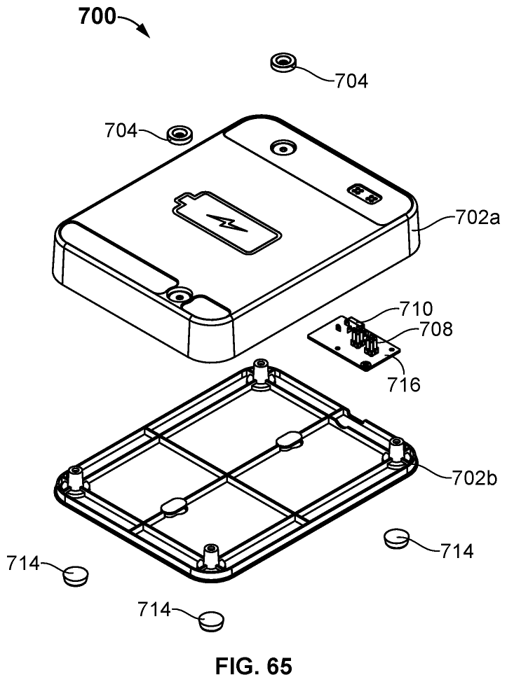

Referring to FIG. 65, an exploded view of the charging station 700 of the UV disinfector 100, according to another embodiment of the present invention. The apparatus 700 has a dimension of about 120×154×27 mm. The apparatus has a charger voltage of about 19V. The apparatus has a weight of about 94 g. In one embodiment, the apparatus 700 comprises a plastic enclosure 702 configured to enclose a plurality of internal components. In one embodiment, the plastic enclosure 702 has at least two parts include a first enclosure part 702a and a second enclosure part 702b. In one embodiment, the apparatus 700 further comprises one or more magnets 704. In one embodiment, the magnets 704 are neodymium magnets. In one embodiment, the apparatus 700 has at least two magnets 704 having the dimension of about 12×3×7-3.5. In one embodiment, the UV disinfector 100 provides a magnetic reinforcement pressure configured to make secure contact with the charging pins.

In one embodiment, the apparatus 700 further comprises a printed circuit board assembly (PCBA) 716. In one embodiment, the PCBA 716 includes a plurality of electrical components operatively mounted on its surface. In one embodiment, the apparatus 700 further comprises at least two spring contact connectors 708 and a power supply connector 710 mounted on the PCBA 716. The apparatus 700 could be installed on a horizontal, inclined surface, and on a wall via the mounting holes 712 using one or more fasteners. In one embodiment, the apparatus 700 is equipped with one or more rubberized pads 714. In one embodiment, the apparatus 700 comprises at least four rubberized pads 714. The rubberized pads 714 are mounted at the bottom corner of the apparatus 700 configured to prevent the apparatus 700 from scratches and other damages.

Preferred embodiments of this innovation are described herein, including the best mode known to the inventors for carrying out the innovation. It should be understood that the illustrated embodiments are exemplary only and should not be taken as limiting the scope of the innovation.

The foregoing description comprises illustrative embodiments of the present innovation. Having thus described exemplary embodiments of the present innovation, it should be noted by those skilled in the art that the within disclosures are exemplary only, and that various other alternatives, adaptations, and modifications may be made within the scope of the present innovation. Merely listing or numbering the steps of a method in a certain order does not constitute any limitation on the order of the steps of that method. Many modifications and other embodiments of the innovation will come to mind to one skilled in the art to which this innovation pertains having the benefit of the teachings in the foregoing descriptions. Although specific terms may be employed herein, they are used only in generic and descriptive sense and not for purposes of limitation. Accordingly, the present innovation is not limited to the specific embodiments illustrated herein.

What is claimed is:

1. An ultraviolet (UV) disinfector for disinfecting an exterior surface, comprising:
a housing having at least two sections include a top section and a base section, wherein the top section and base section are configured to securely fasten together using one or more fasteners;
a controller having a processor and a memory in communication with the processor, wherein the memory is configured to store a set of instructions, which are executed by the processor;
an UV light-emitting diode (LED) array securely affixed to the base section of the housing, wherein the UV light-emitting diode (LED) array in communication with the controller is configured to provide a disinfecting illumination with an ultraviolet light to the exterior surface;
a plurality of sensors in communication of the controller is configured to send one or more signals to the controller, thereby controlling the operation of the UV disinfector;
wherein the UV disinfector is configured to perform one or more functions by the processor, wherein the one or more functions including:
protecting against switching on the UV LED array of in the absence of the surface being treated opposite the radiation port;
automatically delivering an effective dosage of ultraviolet radiation for antibacterial and antiviral treatment of various surfaces by sending control signals to the operator, thereby optimizing the UV disinfector's speed of movement;
optimizing the disinfection process in terms of speed based on a selected mode of treatment depth;
exceptional protecting against the effects of hard UV radiation on the skin surface and eye's retina, and
generating a control file containing fiscal statistical information about the surface treatment carried out for a certain period, compliance with the processing speed and other operating modes of the UV disinfector.

2. The ultraviolet (UV) disinfector of claim 1, is further configured to enable the user to operate in different operating modes based on the surface disinfection.

3. The ultraviolet (UV) disinfector of claim 1, further comprises a mode key, wherein the mode key in communication with the controller is configured to enable the user to select different operating modes.

4. The ultraviolet (UV) disinfector of claim 1, is further configured to optimize the process of disinfection in terms of speed based on the selected mode of treatment depth.

5. The ultraviolet (UV) disinfector of claim 1, is further configured to allow adaptation of the treatment speed according to preset dosage modes.

6. The ultraviolet (UV) disinfector of claim 1, wherein the plurality of sensors is a combination of a proximity sensor, motion speed sensors, an acceleration sensor, a gyro sensor, and a temperature sensor.

7. The ultraviolet (UV) disinfector of claim 1, further comprises a magnetic mount integrated into the UV disinfector for the installation of an additional equipment.

8. The ultraviolet (UV) disinfector of claim 1, further comprises a battery assembly, wherein the battery assembly in communication with the controller is configured to supply electrical power to the UV light-emitting diode (LED) array and the plurality of sensors.

9. The ultraviolet (UV) disinfector of claim 1, further comprises one or more USB ports for enable a user to recharge the power source using a cable.

10. The ultraviolet (UV) disinfector of claim 1, further comprises a reflector, wherein the reflector is securely affixed to the base section in order to increase the utilization ratio of ultraviolet radiation emitted from the UV light-emitting diode (LED) array.

11. The ultraviolet (UV) disinfector of claim 8, wherein the reflector is made of stainless steel.

12. The ultraviolet (UV) disinfector of claim 1, further comprises a LED indicator, wherein the LED indicator is in communication with the controller, configured to indicate the mode of operation selected by the user, wherein the LED indicator is further configured to operate in both color and pulse-information modes.

13. The ultraviolet (UV) disinfector of claim 1, wherein the housing is made of plastic.

14. The ultraviolet (UV) disinfector of claim 1, wherein the housing further comprises one or more ventilation slots for allowing air to flow into and out of the housing, thereby maintaining temperature within the housing of the UV disinfector.

15. The ultraviolet (UV) disinfector of claim 1, further comprises multiple disinfecting apparatus, which are configured to affix or fasten to the UV disinfector for disinfecting small objects, flat surfaces, toroidal surfaces, flat objects with non-uniform surfaces, and cylindrical objects.
